(19) United States
(12) Patent Application Publication
(10) Pub. No.: US 2025/0160878 A1
(43) Pub. Date: May 22, 2025

(54) CATHETERS, DEVICES, AND METHODS FOR REMOVING MATERIALS FROM HOLLOW BODIES

(71) Applicant: Bard Peripheral Vascular, Inc., Franklin Lakes, NJ (US)

(72) Inventors: John Evans, Draper, UT (US); Bradley Forsyth, Buda, TX (US); German Loesener, Saratoga Springs, UT (US)

(73) Assignee: Bard Peripheral Vascular, Inc., Franklin Lakes, NJ (US)

(21) Appl. No.: 18/873,572

(22) PCT Filed: Jun. 24, 2022
(86) PCT No.: PCT/US22/34925

§ 371 Date: Dec. 10, 2024

(51) Int. Cl. A61B 17/3207; A61B 17/32

(52) U.S. Cl. CPC A61B 17/320725; A61B 2017/320044; A61B 2017/320733; A61B 2217/005

(57) ABSTRACT

A catheter for removal of material from hollow bodies is provided. The catheter may include a catheter body defining an opening and a cutting ribbon extending around the catheter body. The cutting ribbon extends out of the opening of the catheter body and has an expanded configuration having a relatively large hoop size and a retracted configuration having a relatively small hoop size. The cutting ribbon includes an elongated ribbon body, and a blade disposed at a side edge of the ribbon body.

### TECHNICAL FIELD

The present disclosure relates to catheters, devices, and methods for removing materials from hollow bodies, and more particularly, catheters, devices, and methods that include a cutting ribbon radially expands to remove materials from hollow bodies.

### BACKGROUND

Materials including plaque may build up in hollow bodies (e.g., blood vessels, such as arteries, and veins). As the materials increase in size, they can reduce the cross sectional area of the hollow bodies. This can lead to congestion or to a total blockage (emboli) of the blood vessel. The health consequences may be circulatory disorders or, in extreme cases, the failure of extremities or even vital organs. It may therefore be useful to have devices removing materials from hollow bodies with an expanding cutting tool.

### SUMMARY

Embodiments of the present disclosure are directed to catheters, devices, and methods that provide an expanding cutting tool, specifically an expanding cutting ribbon, for removing materials from hollow bodies or for endovascular treatment as will be described in greater detail below.

In one embodiment, a catheter for removal of material from hollow bodies is provided. The catheter may include a catheter body defining an opening. A cutting ribbon extends around the catheter body. The cutting ribbon extends out of the opening of the catheter body and having an expanded configuration having a relatively large hoop size and a retracted configuration having a relatively small hoop size. The cutting ribbon includes an elongated ribbon body, and a blade disposed at a side edge of the ribbon body.

In another embodiment, a surgical device is provided. The surgical device may include a catheter for removal of material from hollow bodies. The catheter includes a catheter body defining an opening. A cutting ribbon extends around the catheter body. The cutting ribbon extends out of the opening of the catheter body and having an expanded configuration having a relatively large hoop size and a retracted configuration having a relatively small hoop size. The cutting ribbon includes an elongated ribbon body, and a blade disposed at a side edge of the ribbon body. A guidewire is extendable through the catheter.

In yet another embodiment, a method of using a catheter for removal of material from hollow bodies is provided. The method may include positioning the catheter at a removal site. The catheter may include a catheter body defining an opening. A cutting ribbon extends around the catheter body. The cutting ribbon extends out of the opening of the catheter body and having an expanded configuration having a relatively large hoop size and a retracted configuration having a relatively small hoop size. The cutting ribbon includes an elongated ribbon body, and a blade disposed at a side edge of the ribbon body. A size adjusting mechanism is configured to rotate the cutting ribbon to expand the cutting ribbon radially. The method further includes rotating the cutting ribbon with the size adjusting mechanism to expand the cutting ribbon radially from the catheter body to a size.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a catheter including a cutting ribbon for removing materials from hollow bodies, where the cutting ribbon is retracted, according to one or more embodiments shown and described herein;

FIG. 2A schematically depicts a distal end portion of the catheter of FIG. 1, where the cutting ribbon is expanded, according to one or more embodiments shown and described herein;

FIG. 2B schematically depicts a cross sectional view of the distal end portion of the catheter of FIG. 2A taken along section line 2B-2B, according to one or more embodiments shown and described herein;

FIG. 3A schematically depicts a catheter including a cutting ribbon which is deployable through an opening disposed at a distal end portion of the catheter, where the cutting ribbon is not deployed, according to one or more embodiments shown and described herein;

FIG. 3B schematically depicts the distal end portion of the catheter of FIG. 3A, where the cutting ribbon is deployed, according to one or more embodiments shown and described herein;

FIG. 4A schematically depicts a cutting ribbon including a blade, according to one or more embodiments shown and described herein;

FIG. 4B schematically depicts a cross sectional view of the cutting ribbon of FIG. 4A taken along section line 4B-4B, according to one or more embodiments shown and described herein;

FIG. 4C schematically depicts a cutting ribbon including a serrated blade, according to one or more embodiments shown and described herein;

FIG. 5 schematically depicts a catheter including a plurality of ribbons, according to one or more embodiments shown and described herein;

FIG. 6A schematically depicts the catheter of FIG. 1 advanced through a hollow body, according to one or more embodiments shown and described herein;

FIG. 6B schematically depicts the catheter of FIG. 1 with the cutting ribbon expanded, according to one or more embodiments shown and described herein; and

FIG. 6C schematically depicts the catheter of FIG. 1 removing a material by being moved in a longitudinal direction of the catheter, according to one or more embodiments shown and described herein.

Reference will now be made in greater detail to various embodiments of the present disclosure, some embodiments of which are illustrated in the accompanying drawings. Whenever possible, the same reference numerals will be used throughout the drawings to refer to the same or similar parts.

### DETAILED DESCRIPTION

Embodiments of the present disclosure are directed to catheters, devices, and methods that provide an expanding cutting tool, specifically an expanding cutting ribbon, for removing materials from hollow bodies. For example, a catheter may be place in a blood vessel to remove deposits disposed in the blood vessel. When the catheter is inserted in the blood vessel, the outer diameter of the catheter may be limited in size to protect the blood vessel wall from scratching, stretching, tearing, and the like. At the same time, the cutting tool may be adjustable in size from a retracted configuration to an expanded configuration to remove deposits within the blood vessel. It may therefore be useful to have catheters with an expandable cutting tool. These and additional features and benefits will be described in greater detail herein.

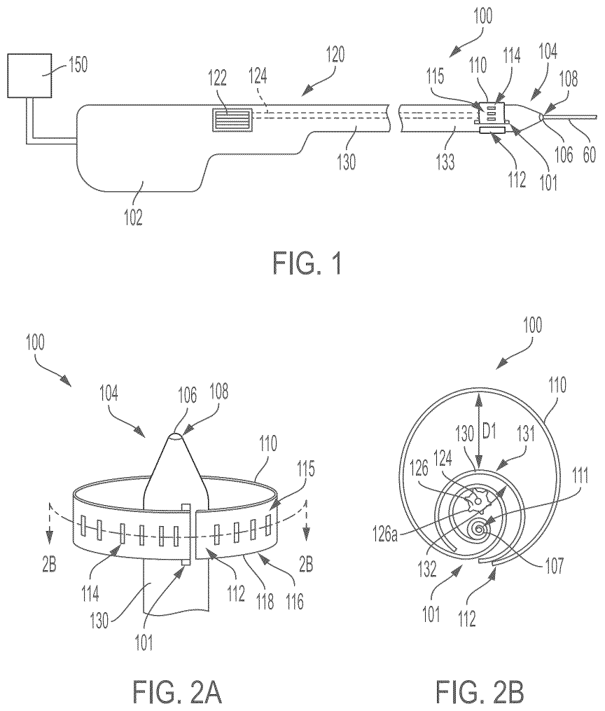

Referring to FIG. 1, a catheter 100 may include a cutting ribbon 110, a size adjusting mechanism 120, and a handle 102. The cutting ribbon 110 may be disposed at a distal end portion 104 of the catheter 100. The catheter 100 has a catheter body 130 which includes a space therein where a portion of the cutting ribbon 110 may be disposed. The catheter body 130 may define an opening 101. The cutting ribbon 110 may extend through the opening 101 formed in a sidewall 133 of the catheter body 130. The cutting ribbon 110 may move between an expanded configuration and a retracted configuration. One end (i.e., a wrapped end 111) of the cutting ribbon 110 may be wrapped around a core 107 located within the catheter body 130. The other end (i.e., a fixed end 112) of the cutting ribbon 110 may be fixed to the catheter body 130, such as an outer surface 131 (FIG. 2B) of the catheter body 130. In other embodiments, the fixed end 112 may re-enter the catheter body 130 at another opening and be fixed to an inner surface 132 (FIG. 2B) of the catheter body 130 or to any other fixed surface or component so that the fixed end does not move. When retracted, the cutting ribbon 110 may be wound against the catheter body 130 and be contiguous with the outer surface 131 of the catheter body 130 when in the retracted configuration. The cutting ribbon 110 may be flexible to be expanded, wrapped, and retracted.

The cutting ribbon 110 may include a plurality of openings 114 positioned along a length of a ribbon body 115 and a blade 116 (FIG. 2A). The cutting ribbon 110 may be operatively coupled to the size adjusting mechanism 120. The size adjusting mechanism 120 may be configured to move the cutting ribbon 110 in a lateral direction of the catheter body 130 to radially expand and retract the cutting ribbon 110. The size adjusting mechanism 120 may include a gear 126 coupled to a shaft 124. Referring also to FIG. 2B, the size adjusting mechanism 120 includes the gear 126 that includes teeth 126a. The teeth 126a may engage with at least one of the plurality of openings 114. For example, the teeth 126a may each be inserted into the at least one of the plurality of openings 114. The openings 114 are aligned along the length of the cutting ribbon 110 so that the teeth 126a may latch with the plurality of openings 114. The blade 116 may be disposed at one side edge 118 of the ribbon body 115, which will be discussed in further detail with reference to FIGS. 2A and 4A-4C.

The shaft 124, while being coupled to the size adjusting mechanism 120, is further coupled to an actuator, such as thumb wheel 122. A user of the catheter 100 may rotate the thumb wheel 122 to expand or retract the cutting ribbon 110. When the thumb wheel 122 is rotated, the shaft 124 rotates to further rotate the cutting ribbon 110 to expand or retract the cutting ribbon 110 away or toward the catheter body 130. The cutting ribbon 110 may expand radially away from the catheter 100. The outer diameter (e.g., loop size) of the cutting ribbon 110 may be adjustable to a certain size by rotating the thumb wheel 122 a selected amount. Adjustable loop sizes may provide size options to a user to select a desired size from the size options, which may adjust cutting potential. Further details of the size adjusting mechanism 120 and the cutting ribbon 110 will be discussed in further detail with reference to FIGS. 2A and 2B.

A distal end 106 of the catheter 100 may be tapered such that the catheter 100 may be smoothly advanced through a hollow body. The distal end 106 may have an opening 108 associated with a lumen extending through the catheter 100. A guide wire 60 may be advanced through the lumen to guide the catheter 100 in the hollow bodies. Suction may be applied by an aspiration device 150 coupled to the catheter 100 to create suction to draw removed material into the lumen of the catheter 100. In some embodiments, the catheter 100 may have a transport mechanism, such as a conveyor, to convey the removed materials through the catheter 100 and collect the materials in a reservoir which may be coupled to the catheter 100. A surgical device may include the catheter 100 and the guide wire to be used together.

In embodiments, an expandable stop may be added as part of the cutting ribbon 110. The expandable stop may act as a stopper for the cutting ribbon 110 such that the cutting ribbon 110 may not touch the hollow body as the materials are being removed. In embodiments, the expandable stop may be a mesh stop allowing blood flows through the expandable stop. In embodiments, the catheter 100 may have an expandable balloon. The expandable balloon may be expanded with saline. The expandable balloon may provide atraumatic pressure against one side of the hollow body, pushing the cutting ribbon 110 against an opposite side of the hollow body.

Referring to FIG. 2A, the cutting ribbon 110 is illustrated in the expanded configuration. The wrapped end 111 of the cutting ribbon 110 may be disposed inside the catheter body 130. The cutting ribbon 110 extends through the opening 101 that is formed in the catheter body 130 and forms a hoop around an outside of the catheter body 130. The fixed end 112 may be fixed to the outer surface 131 adjacent to the opening 101 such that the hoop extends in a continuous manner outward from the opening 101, around the catheter body 130 and back toward the opening 101. The fixed end 112 may be fixed at a different location along the catheter 100 either in the longitudinal direction or a lateral direction around the catheter 100, so long as the cutting ribbon 110 remains expandable.

In some embodiments, the fixed end 112 and the wrapped end 111 of the cutting ribbon 110 may be both disposed inside of the catheter body 130. The fixed end 112 may be extended through the opening 101 and fixed to the inner wall of the catheter body 130. In embodiments, the catheter body 130 may have an additional opening which the fixed end 112 may extend through.

The cutting ribbon 110 may be made with materials that are flexible enough to be wrapped around the core 107 and be expanded to form the hoop. The materials may be rigid enough to retain the shape of the cutting ribbon 110 when expanded, such as a shape-memory material for example. The cutting ribbon 110 may be made from metal such as stainless, nitinol (i.e., a metal alloy of nickel and titanium), or the like, as non-limiting examples. The cutting ribbon 110 may contain elements for visualization such as markers that allow a user to view the cutting ribbon 110 inside the hollow body. The markers may be visualized on a display by an imaging device. The imaging device may provide images of the hollow body and the cutting ribbon 110 to provide information to help the user to select the size of the cutting ribbon 110 or to move the catheter 100 based on the location and/or the position of the cutting ribbon 110, the size of the hollow body, and/or the size of the material.

Referring to FIG. 2B, a cross sectional view of FIG. 2A taken along line 2B-2B is depicted. As described above, the wrapped end 111 is wrapped around the core 107 inside of the catheter 100. The wrapped end 111 may be fixed on the core 107. The core 107 may be rotated when the cutting ribbon 110 is rotated. In some embodiments, the core 107 may be a rod that is rotatably supported within the catheter body 130 and the wrapped end 111 wrapped around the core 107. The core 107 may be coupled to the size adjusting mechanism 120 and be rotated together with or individually from the shaft 124. The cutting ribbon 110 extends from the wrapped end 111 and engages with the gear teeth 126a of the gear 126. In embodiments, the gear 126 may have the gear teeth 126a. The gear teeth 126a may engage with the openings 114 of the cutting ribbon 110. Pitch of the gear teeth 126a may correspond to the distance between the openings 114. When the gear 126 is rotated by the thumb wheel 122 that further rotates the shaft 124, the gear teeth 126a may engage with at least one of the openings 114 and wind or unwind the cutting ribbon 110. For example, rotating the gear 126 clockwise in FIG. 2B may unwind the cutting ribbon 110, and the cutting ribbon 110 may expand as a result. Conversely, rotating the gear 126 counterclockwise in FIG. 2B may wind the cutting ribbon 110, and the cutting ribbon 110 may retract as a result.

The size of an outer diameter of the cutting ribbon 110 and the hoop size of the cutting ribbon 110 may increase or decrease by rotating the gear 126. As used herein, the term “hoop size” refers to the length of the cutting ribbon 110 extending from the opening 101 to the fixed end 112 or the maximum distance D1 from the catheter body 130 to the cutting ribbon 110. As noted above, the gear 126 may be rotated using the thumb wheel 122. In embodiments, the cutting ribbon 110 may be rotated by a motor coupled to the the shaft 124. The motor may be driven by power source, such as electrical or pneumatic. When the motor is utilized, rotational speed of the cutting ribbon 110 may be controlled by either adjusting a user input on the handle 102 or a pedal. The shaft 124 may be coupled to a gearbox to change a torque ratio and/or rotational speed from the thumb wheel 122 or motor to the gear 126 rotating the cutting ribbon 110.

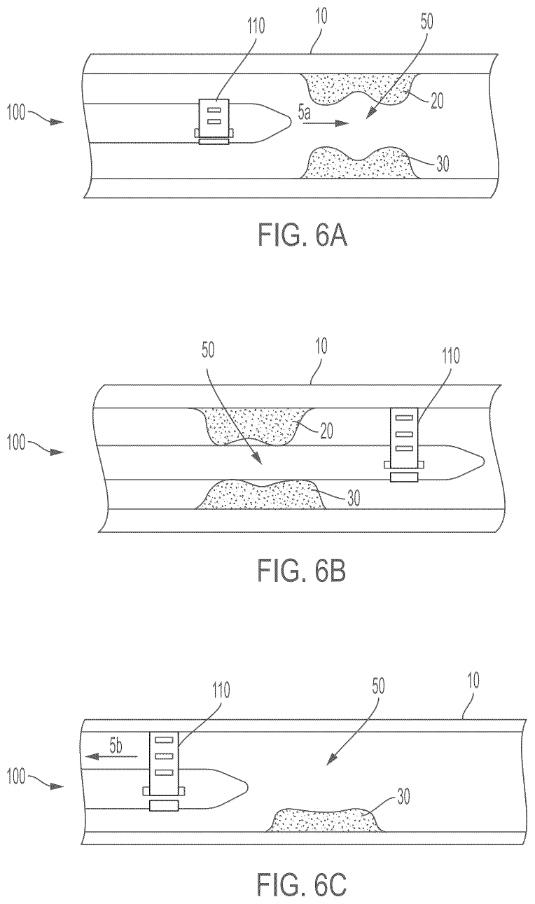

In embodiments, the size of the outer diameter or the hoop of the cutting ribbon 110 may be maintained. For example, once the cutting ribbon 110 is expanded to a desired hoop size, the size may be maintained while the catheter 100 is moved to remove materials in the hollow body. For example, the radial size of the cutting ribbon 110 may be fixed when expanded to a desired size so that the size does not change while conducting removing procedure including moving the catheter 100 in a distal direction 5a (FIG. 6A) or a proximal direction 5b (FIG. 6B), or rotating the catheter 100.

In embodiments, the size adjusting mechanism 120 may provide tactile feedback to a user of the catheter 100 when the gear 126 is rotated. For example, the size adjusting mechanism 120 may provide a clicking sound or vibration when one of the gear teeth 126a engages with one of the openings 114. The tactile feedback may provide the user with information indicative of the hoop size of an outer dimeter of the cutting ribbon 110. In an embodiment, the information indicative of the hoop size may be available through an indicator (e.g., numbers or colors) printed on the thumb wheel 122. In embodiments, the tactile feedback may indicate the cutting ribbon 110 has expanded to touch the hollow body. The user may confirm the size of the outer dimeter of the cutting ribbon 110 based on the tactile feedback.

It is noted that selectable size options of the cutting ribbon 110 provided by the size adjusting mechanism 120 may allow a user of the catheter 100 to select or change the size of the cutting ribbon 110 during a procedure. This may allow the user to promptly respond to unexpected situations during a procedure. For example, when unexpected materials are present in the hollow body, the user may remove the unexpected materials during a procedure for removing target materials, which may reduce the total number of procedures. The size adjustable cutting ribbon 110 may still be adaptable or modifiable to remove the material accordingly even in a situation where a target material may be bigger, harder, or fibrous than expected.

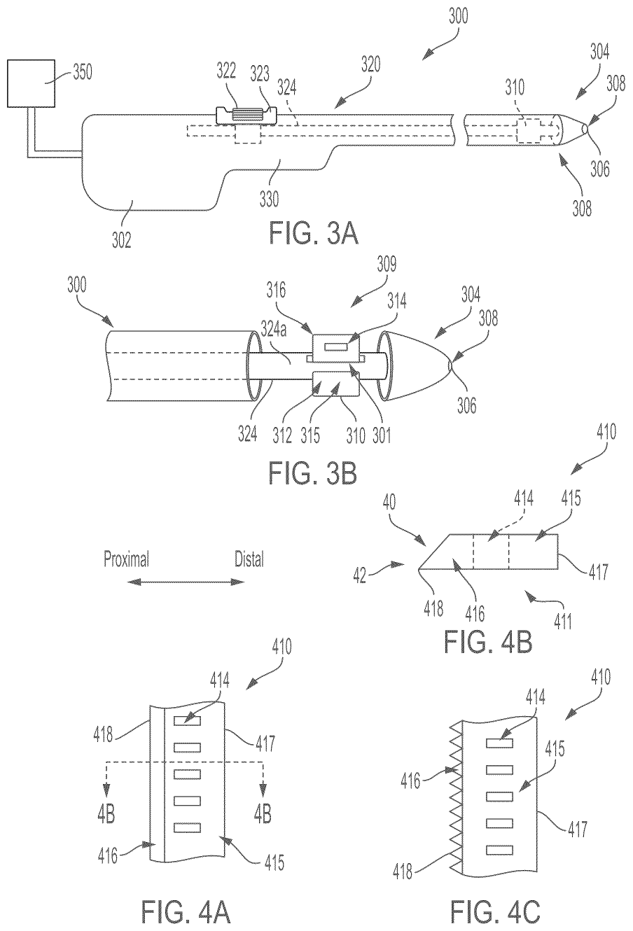

FIG. 3A-3B illustrate embodiments of a catheter 300 including a cutting ribbon 310 disposed inside of the catheter 300. The catheter 300 is generally similar to the catheter 100 illustrated in FIGS. 1 and 2A-2B unless otherwise noted.

Referring to FIG. 3A, the catheter 300 may include a cutting ribbon 310, a size adjusting mechanism 320, and a handle 302. The catheter 300 has a catheter body 330 which includes a space in which the cutting ribbon 310 may be disposed. The cutting ribbon 310 may be disposed at a distal end portion 304 of the catheter body 330. The cutting ribbon 310 may be deployable through an opening 309 formed in the catheter body 330. The cutting ribbon 310 may be deployed by being moved in a longitudinal direction of the catheter 300. The opening 309 is obtained by moving or pushing the shaft 324 in a distal direction of the catheter 300. The distal end portion 304 is also moved in the distal direction relative to the catheter body 330 when the shaft 324 is moved in the distal direction. When the opening 309 is obtained, the cutting ribbon 310 is deployable through the opening 309. When deployed, the cutting ribbon 310 may be rotated to be expanded or retracted through the opening 309.

The cutting ribbon 310 may extend through an opening 301 formed in a side wall of a shaft 324. The cutting ribbon 310 may be extended or retracted through the opening 301. Further, one end (i.e., a wrapped end 311) of the cutting ribbon 310 may be wrapped inside of the shaft 324 (e.g., around a core similar to the core 107 in FIG. 2B). The other end (i.e., a fixed end 312) of the cutting ribbon 310 may be fixed to the shaft 324, such as an outer surface 324a of the shaft 324. In other embodiments, the fixed end 312 may re-enter the shaft 324 at another opening and be fixed to an inner surface of the shaft 324 or to any fixed surface or component so that the fixed end 312 does not move. When retracted, the cutting ribbon 310 may be wound against the outer surface of the shaft 324. The shaft 324 may be axially rotated or longitudinally moved with respect to the catheter body 330. The cutting ribbon 310 may be flexible to be expanded, wrapped, and retracted. The cutting ribbon 310 may include a plurality of openings 314 positioned along a length of a ribbon body 315 and a blade 316.

The size adjusting mechanism 320 of the catheter 300 may include a deploy mechanism 323 (e.g., thumb clamp) to extend the shaft 324 and deploy the cutting ribbon 310 from the catheter body 330. The cutting ribbon 310 may be deployed through the opening 309. The deploy mechanism 323 is coupled to the cutting ribbon 310 via the shaft 324. In some embodiments, the deploy mechanism 323 may be spring loaded. In some embodiments, a user may move (e.g., slide) the deploy mechanism 323 to slide out the shaft 324 and the cutting ribbon 310 from the catheter 300.

Referring to FIG. 3B, the cutting ribbon 310 is deployed. The cutting ribbon 310 may include a plurality of openings 314 that may engage with a gear (similar to gear 126 with gear teeth 126a discussed with reference to FIG. 2B) coupled to an inner shaft. The gear may be coupled to the inner shaft directly or indirectly. The inner shaft is further coupled to an actuator, such as a thumb wheel 322. A user of the catheter 300 may rotate the thumb wheel 322 to expand or retract the cutting ribbon 310 in a fashion similar to that described above. When the thumb wheel 322 is rotated, the inner shaft rotates to further rotate the cutting ribbon 310 to expand the cutting ribbon 310. The cutting ribbon 310 may expand radially from the shaft 324 and the catheter 300. The outer diameter (e.g., loop size) of the cutting ribbon 310 may be adjustable to a certain size by rotating the thumb wheel 322. Adjustable hoop sizes may provide size options to a user to choose desired size from the size options, which may maximize cutting potential. Further details of the size adjusting mechanism 320 and the cutting ribbon 310 are similar to the size adjusting mechanism 120 and the cutting ribbon 110 in FIGS. 2A and 2B, except at least that the fixed end 312 of the cutting ribbon 310 is fixed on the shaft 324 instead of the catheter body 330, and unless otherwise noted.

A distal end 306 of the catheter 300 may be tapered such that the catheter 300 may be advanced through hollow bodies smoothly. The distal end 306 may have an opening 308 for a lumen extending through the catheter 300. A guidewire may be advanced through the lumen to guide the catheter 300 in the hollow bodies. Suction may be applied by an aspiration device 350 coupled to the catheter 300 to create suction to draw removed materials into the lumen of the catheter 300. The catheter 300 may have a transport mechanism to convey the removed materials through the catheter 300 and collect them in a reservoir which may be coupled to the catheter 300 to collect the removed materials.

In embodiments, an expandable stop may be added as part of the cutting ribbon 310. The expandable stop may act as a stopper for the cutting ribbon 310 such that the cutting ribbon 310 may not touch the hollow body as the materials are being removed. In embodiments, the expandable stop may be a mesh stop allowing blood flows through the expandable stop. In embodiments, the catheter 300 may have an expandable balloon. The expandable balloon may be expanded with saline. The expandable balloon may provide atraumatic pressure against one side of the hollow body, pushing the cutting ribbon 310 against an opposite side of the hollow body.

FIGS. 4A-4C are various embodiments of a cutting ribbon 410. Referring to FIG. 4A, the cutting ribbon 410 may have a ribbon body 415 and a blade 416 disposed at least one of side edges 417, 418 of the ribbon body 415. The ribbon body 415 has a distal side edge 417 disposed on the distal side of the longitudinal direction of a catheter, and a proximal side edge 418 disposed on the proximal side of the longitudinal direction of a catheter.

In embodiments, the blade 416 may be disposed on at least one of the proximal side edge 418 or the distal side edge 417 or both. FIG. 4A depicts the cutting ribbon 410 having the blade 416 disposed on the proximal side edge 418. When the blade 416 is disposed on the proximal side edge 418, moving the cutting ribbon 410 in a proximal direction may cut materials disposed in the proximal side of the cutting ribbon 410. Conversely, when the blade 416 is disposed on the distal side edge 417, moving the cutting ribbon 410 in a distal direction may cut materials disposed in the distal side of the cutting ribbon 410.

When one or both of the side edges 417, 418 do not constitute a blade, the side edges 417, 418 may be blunt. The term “blunt” refers to a structure that is not sharp, dull, lacking sharp corners, and for example, refers to a structure that is round, obtuse, or flat. When blunt, the side edges 417, 418 may be round edges, straight edges, or the like. When blunt, the side edges 417, 418 may push or provide pressure against materials in the hollow bodies to change shape or location of the materials.

Referring to FIG. 4B is a cross sectional view of the cutting ribbon 410 taken along line 4B-4B in FIG. 4A. The blade 416 may have a bevel 40 facing away from the catheter body forming a blade edge 42 located at an interior side 411 of the cutting ribbon 410. When the cutting ribbon 410 is placed in a hollow body, the bevel 40 may face an inner wall of the hollow body such that the blade 416 is spaced from the hollow body. The blade 416 may have a double bevel or different shapes so long as the blade edge 42 is spaced from with the blade 416 in contact with an inner surface of the hollow body. The blade 416 may be curved toward the catheter such that the blade edge 42 faces down toward the catheter (i.e., faces away from a hollow body). Sharpness of the blade 416 may be designed to be sharp enough to remove materials from the hollow body by cutting or scraping the materials out of the hollow body, but not sharp enough to cut through the hollow body.

Referring to FIG. 4C, the blade 416 may be serrated to enhance cutting properties (e.g., cutting power) of the blade 416. The blade 416 may be scalloped, waved, double serrated, or the like. When the blade 416 is serrated, the blade 416 may catch, rip, shred, or tear the materials to be removed that may enhance cutting power of the blade 416.

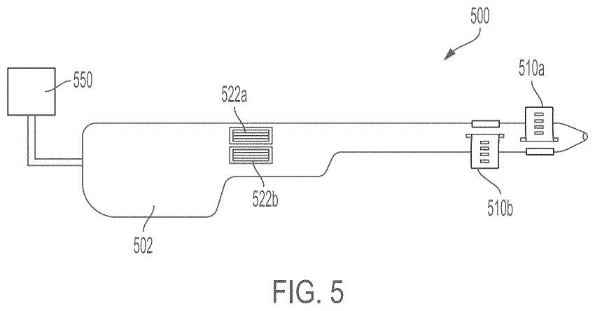

In embodiments, referring to FIG. 5, the catheter 500 may have a plurality of ribbons 510a, 510b. The ribbons 510a, 510b may be disposed along the longitudinal direction of the catheter 500. The ribbons 510a, 510b may include at least one cutting ribbon and one blunt ribbon. In embodiments, one of the ribbon 510a may be a blunt cutting ribbon and the ribbon 510b may be a cutting ribbon. The cutting ribbon may have at least one side edge having a blade. In embodiments, one of the ribbon 510a and the ribbon 510b may be a blunt ribbon. The blunt ribbon 510a may have both side edges blunt. In such case, the blunt ribbon 510a may push or provide pressure against a hollow body away from the catheter 100 so that to create space between the catheter 100 and the hollow body.

In embodiments, the blunt ribbon 510a may be expanded toward one side of the hollow body, while the cutting ribbon 510b may be expanded toward an opposite side of the hollow body where materials to be removed are disposed. In such case, the cutting ribbon 510b may be pushed against the opposite side of the hollow body and the materials. Therefore, the blunt ribbon 510a may assist the cutting ribbon 510b removing the materials by pushing the cutting ribbon 510b toward the materials.

In embodiments, both the blunt ribbon 510a and the cutting ribbon 510b may be expanded toward the same side of the hollow body where materials to be removed are disposed. The blunt ribbon 510a may create or increase a space between the catheter 500 and the hollow body. The space created by the blunt ribbon 510a may provide enough space for the cutting ribbon 510b to expand. The space may prevent inadvertent contact with the hollow body.

In embodiments, two or more ribbons may be cutting ribbons. For example, both of the ribbon 510a and the ribbon 510b may be cutting ribbons. Each of the cutting ribbons 510a and the ribbon 510b may have at least one side edge having a blade. The two or more cutting ribbons 510a, 510b configuration may increase cutting power. The cutting ribbons 510a, 510b may be disposed to expand in different directions. For example, one cutting ribbon 510a, 510b may expand toward one side of the hollow body and the other cutting ribbon 510a, 510b may expand toward an opposite side of the hollow body. In such case, the cutting ribbons may provide cutting of multiple materials disposed in different locations within one procedure.

In embodiments, the two or more cutting ribbons 510a, 510b may be disposed to expand in the same direction. For example, both of the ribbon 510a and the ribbon 510b may expand toward the same side of the hollow body.

FIGS. 6A-6C depicts a process of removing materials 20, 30 from a hollow body 10 using the catheter 100. It is noted that similar procedure may be utilized for the catheter 300, the catheter 500, or in various embodiments.

Referring to FIG. 6A, the catheter 100 is advanced through a hollow body 10 in a distal direction 5a and approaches a removal site 50 where materials 20, 30 are present.

Referring to FIG. 6B, the catheter 100 is further advanced through the hollow body 10 and positioned at the removal site 50. When positioned at the removal site 50, the cutting ribbon 110 may be positioned at a distal side of a location of the materials 20, 30. A user may expand the cutting ribbon 110 by utilizing the size adjusting mechanism 120. For example, the user may rotate the thumb wheel 122 (FIG. 1) to rotate the gear 126 inside the catheter body 130, which expands the cutting ribbon 110. When the cutting ribbon 110 is expanded, the user may receive tactile feedback which may provide size information of the cutting ribbon 110. For example, one click or vibration may indicate the cutting ribbon 110 is expanded for one pitch of the openings 114. The user may feel clicking or vibration from the size adjusting mechanism 120 through the catheter 100 or the handle 102. For example, fitting between the gear teeth 126a (FIG. 2B) and one of the openings 114 may create vibration or clicking, or the catheter 100 may have additional clicking mechanism to provide tactile feedback when the thumb wheel 122 is rotated. In embodiments, the tactile feedback may indicate the cutting ribbon 110 has expanded to be reached a certain size that may touch the hollow body 10. The size of the cutting ribbon 110 may be maintained after being expanded.

After the cutting ribbon 110 is expanded, referring to FIG. 6C, the catheter 100 may be moved in a proximal direction 5b to cut or push at least one of the materials 20, 30 to remove the materials 20, 30 from the removal site 50. This movement in the proximal direction 5b is similar to dragging the catheter 100 back through the removal site 50.

In embodiments, the catheter 100 may be positioned at the removal site 50 where the cutting ribbon 110 may be positioned at a proximal side of the removal site 50 (e.g., the location of the materials 20, 30). In case where the cutting ribbon 110 is positioned at the proximal side, the catheter 100 may be moved in the distal direction 5a to cut or push at least one of the materials 20, 30 to remove from the removal site 50.

When the catheter 500 (FIG. 5) including the plurality of ribbons 510a, 510b, is utilized, one of the ribbons 510a, 510b may be expanded first and the other one of the ribbons 510a, 510b expanded later. The ribbon expanded first may push or provide pressure against the inner wall of the hollow body 10 to assist the other ribbon expanded later. The ribbon first expanded may be a blunt ribbon with a blunt edge and the ribbon later expanded may be a cutting ribbon with a blade. The ribbon first expanded may be a cutting ribbon with a blade and the ribbon later expanded may be a blunt ribbon. Both of the ribbons may be cutting ribbons or blunt ribbons.

The radial size of the cutting ribbon 110 may be fixed when expanded to a desired size so that the size does not change while conducting removing procedure including moving the catheter 100 in the distal direction 5a or the proximal direction 5b, or rotating the catheter 100. The cutting ribbon 110 may be moved back and forth in the distal direction 5a and the proximal direction 5b.

The catheter 100 may be rotated such that the cutting ribbon faces the other side of the hollow body 10 where a plurality of materials are present. In embodiments, the material 20 may be removed first and the catheter 100 may be rotated to position the cutting ribbon 110 to face toward the material 30. The material 30 may be removed by advancing the catheter 100 in the distal direction 5a or in the proximal direction 5b. In embodiments, the cutting ribbon 110 may be retracted after removing the material 20 and then rotated to reposition the cutting ribbon 110 facing toward the material 30. Then, the cutting ribbon 110 is expanded to remove the material 30. The process may be repeated until all the materials are removed from the hollow body 10. The cutting ribbon 110 may be retracted after finishing the removing procedure to be taken out from the hollow body 10. The removed materials 20, 30 may be vacuumed through the catheter 100 to be taken out from the hollow body 10.

Embodiments can be described with reference to the following numerical clauses:

1. A catheter for removal of material from hollow bodies, comprising: a catheter body defining an opening; a cutting ribbon extending around the catheter body, the cutting ribbon extending out of the opening of the catheter body and having an expanded configuration having a relatively large hoop size and a retracted configuration having a relatively small hoop size, the cutting ribbon comprising; an elongated ribbon body, and a blade disposed at a side edge of the ribbon body.

2. The catheter of clause 1, further comprising a size adjusting mechanism configured to rotate the cutting ribbon to expand the cutting ribbon radially.

3. The catheter of clause 2, wherein the ribbon body defines a plurality of openings positioned along a length of the ribbon body, and the size adjusting mechanism includes a gear configured to engage the plurality of openings.

4. The catheter of any one of clauses 2 to 3, wherein the size adjusting mechanism is configured to provide tactile feedback to a user to confirm a size of an outer diameter of the cutting ribbon.

5. The catheter of any one of clauses 2 to 4, wherein the size adjusting mechanism is configured to maintain an outer diameter of the cutting ribbon when the cutting ribbon is expanded.

6. The catheter of any preceding clauses, wherein the blade has a bevel facing away from the catheter body.

7. The catheter of clause 6, wherein the blade is serrated.

8. The catheter of any preceding clauses, wherein the cutting ribbon has terminal ends, one of the terminal ends is disposed inside the catheter body.

9. The catheter of any preceding clauses, wherein the opening is disposed in a sidewall of the catheter body.

10. The catheter of any preceding clauses, wherein the opening is disposed in a distal end portion of the catheter body.

11. The catheter of any preceding clauses, further comprising multiple ribbons including the cutting ribbon.

12. A surgical device, comprising: a catheter for removal of material from hollow bodies comprising, a catheter body defining an opening, a cutting ribbon extending around the catheter body, the cutting ribbon extending out of the opening of the catheter body and having an expanded configuration having a relatively large hoop size and a retracted configuration having a relatively small hoop size, the cutting ribbon comprising an elongated ribbon body, and a blade disposed at a side edge of the ribbon body; and a guidewire extendable through the catheter.

13. The surgical device of clause 12, further comprising a size adjusting mechanism configured to rotate the cutting ribbon to expand the cutting ribbon radially.

14. The surgical device of clause 13, wherein the ribbon body defines a plurality of openings positioned along a length of the ribbon body, and the size adjusting mechanism includes a gear configured to engage the plurality of openings.

15. The surgical device of any preceding clauses, wherein the blade has a bevel facing away from the catheter body.

16. The surgical device of any preceding clauses, further comprising an aspiration device coupled to the catheter to create suction to draw removed material into a lumen of the catheter.

17. A method of using a catheter for removal of material from hollow bodies, comprising: positioning the catheter at a removal site, the catheter comprising, a catheter body defining an opening; a cutting ribbon extending around the catheter body, the cutting ribbon extending out of the opening of the catheter body and having an expanded configuration having a relatively large hoop size and a retracted configuration having a relatively small hoop size, the cutting ribbon comprising an elongated ribbon body, and a blade disposed at a side edge of the ribbon body; and a size adjusting mechanism configured to rotate the cutting ribbon to expand the cutting ribbon radially; and rotating the cutting ribbon with the size adjusting mechanism to expand the cutting ribbon radially from the catheter body to a size.

18. The method of clause 17, further comprising: fixing the size of the cutting ribbon; and moving the cutting ribbon in a longitudinal direction of the catheter.

19. The method of any preceding clauses, further comprising rotating the cutting ribbon to retract in the catheter body.

20. The method of any preceding clauses, further comprising rotating a blunt ribbon to expand radially, wherein the catheter includes the blunt ribbon having a blunt edge, and the blunt ribbon is radially expandable.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A catheter for removal of material from hollow bodies, comprising:
a catheter body defining an opening;
a cutting ribbon extending around the catheter body, the cutting ribbon extending out of the opening of the catheter body and having an expanded configuration having a relatively large hoop size and a retracted configuration having a relatively small hoop size, the cutting ribbon comprising;
an elongated ribbon body, and
a blade disposed at a side edge of the ribbon body.

2. The catheter of claim 1, further comprising a size adjusting mechanism configured to rotate the cutting ribbon to expand the cutting ribbon radially.

3. The catheter of claim 2, wherein the ribbon body defines a plurality of openings positioned along a length of the ribbon body, and the size adjusting mechanism includes a gear configured to engage the plurality of openings.

4. The catheter of claim 2, wherein the size adjusting mechanism is configured to provide tactile feedback to a user to confirm a size of an outer diameter of the cutting ribbon.

5. The catheter of claim 2, wherein the size adjusting mechanism is configured to maintain an outer diameter of the cutting ribbon when the cutting ribbon is expanded.

6. The catheter of claim 1, wherein the blade has a bevel facing away from the catheter body.

7. The catheter of claim 6, wherein the blade is serrated.

8. The catheter of claim 1, wherein the cutting ribbon has terminal ends, one of the terminal ends is disposed inside the catheter body.

9. The catheter of claim 1, wherein the opening is disposed in a sidewall of the catheter body.

10. The catheter of claim 1, wherein the opening is disposed in a distal end portion of the catheter body.

11. The catheter of claim 1, further comprising multiple ribbons including the cutting ribbon.

12. A surgical device, comprising:
a catheter for removal of material from hollow bodies comprising,
a catheter body defining an opening,
a cutting ribbon extending around the catheter body, the cutting ribbon extending out of the opening of the catheter body and having an expanded configuration having a relatively large hoop size and a retracted configuration having a relatively small hoop size, the cutting ribbon comprising an elongated ribbon body, and a blade disposed at a side edge of the ribbon body; and
a guidewire extendable through the catheter.

13. The surgical device of claim 12, further comprising a size adjusting mechanism configured to rotate the cutting ribbon to expand the cutting ribbon radially.

14. The surgical device of claim 13, wherein the ribbon body defines a plurality of openings positioned along a length of the ribbon body, and the size adjusting mechanism includes a gear configured to engage the plurality of openings.

15. The surgical device of claim 12, wherein the blade has a bevel facing away from the catheter body.

16. The surgical device of claim 12, further comprising an aspiration device coupled to the catheter to create suction to draw removed material into a lumen of the catheter.

17. A method of using a catheter for removal of material from hollow bodies, comprising:
positioning the catheter at a removal site, the catheter comprising,
a catheter body defining an opening;
a cutting ribbon extending around the catheter body, the cutting ribbon extending out of the opening of the catheter body and having an expanded configuration having a relatively large hoop size and a retracted configuration having a relatively small hoop size, the cutting ribbon comprising an elongated ribbon body, and a blade disposed at a side edge of the ribbon body; and
a size adjusting mechanism configured to rotate the cutting ribbon to expand the cutting ribbon radially; and
rotating the cutting ribbon with the size adjusting mechanism to expand the cutting ribbon radially from the catheter body to a size.

18. The method of claim 17, further comprising:
fixing the size of the cutting ribbon; and
moving the cutting ribbon in a longitudinal direction of the catheter.

19. The method of claim 17, further comprising rotating the cutting ribbon to retract in the catheter body.

20. The method of claim 17, further comprising rotating a blunt ribbon to expand radially, wherein the catheter includes the blunt ribbon having a blunt edge, and the blunt ribbon is radially expandable.
